(19) United States
(12) Patent Application Publication
(10) Pub. No.: US 2020/0179968 A1
(43) Pub. Date: Jun. 11, 2020

(54) LIQUID GAS OVERSPRAY PROTECTION SHIELD

(71) Applicant: John Kniffen, San Antonio, TX (US)

(72) Inventor: John Kniffen, San Antonio, TX (US)

(21) Appl. No.: 16/216,922

(22) Filed: Dec. 11, 2018

(51) Int. Cl. B05B 12/20

(52) U.S. Cl. CPC B05B 12/20; A61B 18/02

(57) ABSTRACT

A protection shield for preventing liquid gas from contacting healthy skin has a first shield, an overlay shield, and a coupler. The first shield has a plurality of openings and a first shield center opening. The overlay shield has an overlay opening and a center opening. The overlay shield is removably coupled to the first shield and prevents overspray of a liquid gas from contacting healthy skin. A coupler locks the first shield and the overlay shield in place.

### FIELD OF THE INVENTION

This invention relates to protection shields. More particularly, it relates to protection shields for liquid gas overspray.

### BACKGROUND

“Cryotherapy,” is comprised of two Greek words “cryo” and “therapeia.” “Cryo” means cold, and “therapeia” which is “therapy” translates to “cure.” Though the basic healing properties behind cold cure are the same, cryotherapy as it is known today has evolved significantly over the years.

The Edwin Smith Papyrus, an ancient Egyptian text (1600 BCE) discussing trauma and trauma treatments (surgical and otherwise), referenced to the use of cold to treat inflammation and injury on multiple pages. In 400 BCE, Hippocrates used cold to relieve swelling and pain. During 1050 AD, Anglo-Saxon Monks began using cold as a local anesthetic. From 1845 to 1851, Dr. James Arnott noted the benefits of applying cold treatment for headaches and neuralgia.

Arnott also used low temperature solutions comprised of salt and crushed ice to freeze various tumors. He observed that the tumors shrunk and the pain accompanying them had decreased. In 1851, Arnott exhibited an apparatus at the Great Exhibition in London designed to take the cold cure further, but the design was complicated and had limited freezing capability. However, Arnott had recognized the analgesic (painkilling) effects of cold, and recommending that it be used as an anesthetic before surgery. Dr. William Pusey introduced solidified carbon dioxide into clinical use and had success in treated warts, vascular nevi, lupus vulgaris and lupus erythematosus, and epitheliomas. Pusey's use of solidified carbon dioxide put liquid air out of use, and after 1910, solid carbon dioxide became the most popular cryogenic agent.

In the 1920s, liquid oxygen went clinical. Dr. Irving and Dr. Turnacliff had found that liquid oxygen successfully treated similar skin conditions as ones treated using others cryogenic substances. However, liquid oxygen was highly combustible.

After World War II, liquid nitrogen went to the market and became commercially available. Dr. Ray Allington brought it to clinical use and would treat a variety of skin diseases using cotton swab application. In 1961, Dr. Irving S. Cooper developed a modern cryosurgical apparatus along with engineer Arnold Lee, which became the prototype for every liquid nitrogen probe to come. In 1967, Setrag Zacarian introduces a condensed liquid nitrogen apparatus, a hand-held, self-pressurized spray device used for localized treatment.

Though liquid nitrogen is effective, it is also prone to be hard to direct the spray only to a given area. Thus, other areas of the skin are subject to the liquid nitrogen spray although they are not the directed at skin. Thus, it is useful to have a protective mechanism for the skin that surrounds a given issue area that is subject and benefits from liquid nitrogen, thus the overspray is a concern for those using liquid nitrogen spray for cryotherapy.

### BRIEF DESCRIPTION OF THE DRAWINGS

FIG. 1 is an illustrated view of the components of the exemplary protection shield.

FIG. 2 illustrates the exemplary protective shield use on human skin.

### DETAILED DESCRIPTION

The phrases “in one embodiment,” “in various embodiments,” “in some embodiments,” and the like are used repeatedly. Such phrases do not necessarily refer to the same embodiment. The terms “comprising,” “having,” and “including” are synonymous, unless the context dictates otherwise. Such terms do not generally signify a closed list.

“Above,” “adhesive,” “affixing,” “any,” “around,” “both,” “bottom,” “by,” “comprising,” “consistent,” “customized,” “enclosing,” “friction,” “in,” “labeled,” “lower,” “magnetic,” “marked,” “new,” “nominal,” “not,” “of,” “other,” “outside,” “outwardly,” “particular,” “permanently,” “preventing,” “raised,” “respectively,” “reversibly,” “round,” “square,” “substantial,” “supporting,” “surrounded,” “surrounding,” “threaded,” “to,” “top,” “using,” “wherein,” “with,” or other such descriptors herein are used in their normal yes-or-no sense, not as terms of degree, unless context dictates otherwise.

Reference is now made in detail to the description of the embodiments as illustrated in the drawings. While embodiments are described in connection with the drawings and related descriptions, there is no intent to limit the scope to the embodiments disclosed herein. On the contrary, the intent is to cover all alternatives, modifications and equivalents. In alternate embodiments, additional devices, or combinations of illustrated devices, may be added to, or combined, without limiting the scope to the embodiments disclosed herein.

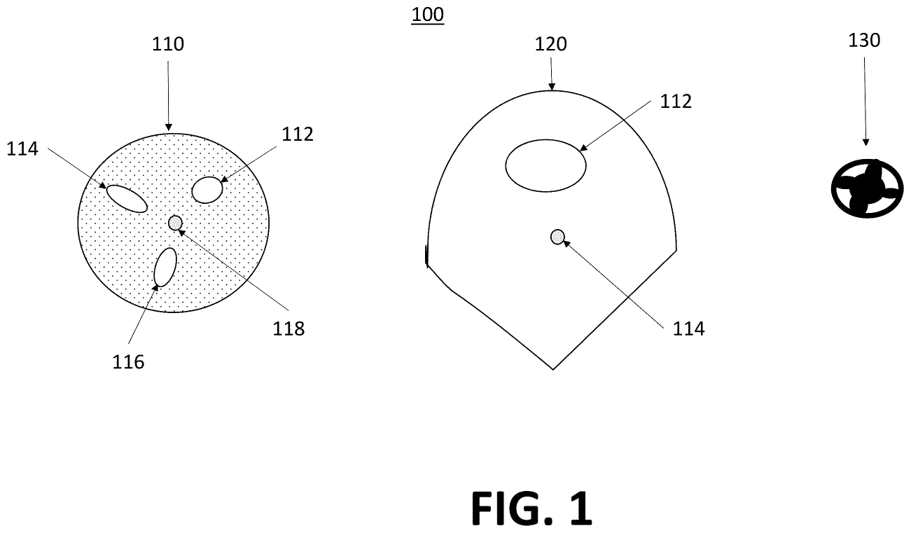

Referring to FIG. 1, components of an exemplary protection shield 100 when applying liquid gas, such as liquid nitrogen, while performing cryotherapy is presented. The protective shield 100 is useful to direct the liquid gas to the troubled area and to prevent the liquid gas from contacting healthy skin. The liquid gas is of such a low temperature that it is used to freeze troubled skin, but in turn, can kill healthy skin in a worst case but in any case causes collateral discomfort.

The protection shield 100 has a first shield 110, an overlay shield 120, and a coupler 130. The first shield 110 has a plurality of openings 112, 114, 116 and a first shield center opening 118. The plurality of openings 112, 114, 116 vary in size to accommodate a troubled area that is being treated. The first shield is preferable plastic, but may be any material which prevents liquid gas from penetrating to a non-treatment area.

Further, the first shield is preferable one twenty five thousands inches (0.125″) in thickness, three to four inches (3-4″) in length and three to four inches (3-4″) wide. The size and shape of the first shield 110 may be any size and shape desirable though the preferable size given above is easy to maneuver and keep stable during treatment.

The overlay shield 120 is useful for covering non-directed to openings 114, 116 of the first shield 110. Thus, when spraying the liquid gas, the overspray is caught on the overlay shield 120 and prevents the liquid gas from making contact with skin not subject to treatment and prevents the overspray from coming in contact with skin in the unused openings 114, 116 of the first shield.

The overlay shield 120 is preferable round but may be any shape that is desirable. The size of the overlay shield 120 is preferable slightly larger than the first shield 110 but not significantly larger. Further, the overlay shield is preferably plastic but may be any material that is nonporous to prevent leakage of the liquid gas to undesired areas.

The overlay shield 120 has an overlay opening 122 and a center opening 124. The center opening 124 of the overlay shield 120 is preferably the same size as the center opening 118 of the first shield. The center opening 124 of the overlay shield 120 is useful to have a coupler 130 to lock the first shield 110 and the overlay shield in place.

The coupler 130 fits through the center opening 124 of the overlay shield 120 and then through a first shield center opening 118 of the first shield 110 and locks the first shield 110 and the overlay shield 120 in unison.

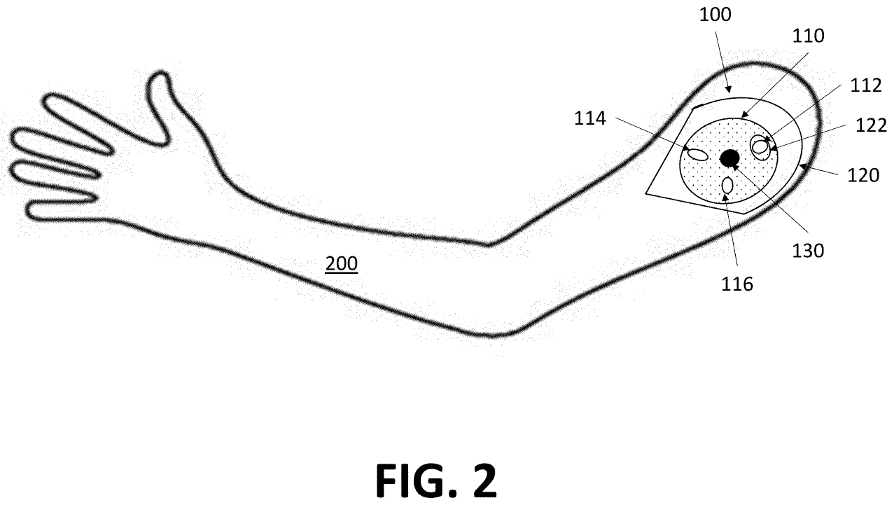

Moving now to FIG. 2, the exemplary protective shield 100 use on a person's skin is illustrate. The arm 200 of a person is shown and has been diagnosed with a troubled area that needs cryotherapy. The troubled area is on the upper arm of the person. The cryotherapy will consists of spraying a liquid gas, such as liquid nitrogen to treat the troubled area. The person desires to not have other skin come in contact with the overspray of the liquid gas to avoid additional discomfort and/or pain.

The person places the first shield 110 on their skin and has an appropriate opening 112 over the troubled area and has other openings 114, 116 of the first shield 110 not over the troubled area. Once the first shield 110 has been placed in the desired position, the overlay shield 120 is placed over the first shield 110 such that the overlay opening 122 of the overlay shield 120 over only the opening 112 such that no other opening 114, 116 are not exposed to the liquid gas overspray.

The coupler 130 is threaded through the center opening 124 of the overlay shield 120 and then through the center opening 118 of the first shield 110. Thus, the first shield 110 and the overlay shield 120 are locked.

The person then sprays the liquid gas on the desired area through overlay opening 122 of the overlay shield 120. The liquid gas then passes through the opening 112 of the first shield 110 and comes into contact with the troubled area. Overspray does not enter the other openings 114, 116 of the first shield and make contact with other skin areas because the overlay shield 120 has no other overlay openings and the spray is captured on the surface of the overlay shield 120.

In the numbered clauses below, specific combinations of aspects and embodiments are articulated in a shorthand form such that (1) according to respective embodiments, for each instance in which a “component” or other such identifiers appear to be introduced (with “a” or “an,” e.g.) more than once in a given chain of clauses, such designations may either identify the same entity or distinct entities; and (2) what might be called “dependent” clauses below may or may not incorporate, in respective embodiments, the features of “independent” clauses to which they refer or other features described above.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

The features described with respect to one embodiment may be applied to other embodiments or combined with or interchanged with the features of other embodiments, as appropriate, without departing from the scope of the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A protection shield for preventing liquid gas from contacting healthy skin, said protection shield comprising:
a first shield having a plurality of openings and a first shield center opening;
an overlay shield, said overlay shield having an overlay opening and a center opening and wherein said overlay shield is removably coupled to said first shield and configured to prevent overspray of said liquid gas from contacting said healthy skin; and
a coupler; said coupler removably attached to said center opening of said overlay shield and said first shield center opening and configured to lock said first shield and said overlay shield in place.

2. The protection shield of claim 1, wherein said plurality of openings of said first shield vary in size to accommodate a troubled area being treated.

3. The protection shield of claim 1, wherein said first shield is made of a plastic material.

4. The protection shield of claim 1, wherein said first shield is approximately 0.125 inches in thickness.

5. The protection shield of claim 1, wherein said first shield is approximately 3 inches to 4 inches in length.

6. The protection shield of claim 1, wherein said first shield is approximately 3 inches to 4 inches in width.

7. The protection shield of claim 1, wherein said overlay shield is round in shape.

8. The protection shield of claim 1, wherein said overlay shield is larger than said first shield.

9. The protection shield of claim 1, wherein said overlay shield is made of a plastic material.

10. The protection shield of claim 1, wherein said center opening of said overlay shield is approximately the same size as said first shield center opening.
